(19) United States
(12) Patent Application Publication
(10) Pub. No.: US 2008/0178715 A1
(43) Pub. Date: Jul. 31, 2008

(54) Needle holder with magnet

(76) Inventor: Orhan Seyfi Aksakal, Ankara (TR)

Correspondence Address: Oppedahl Patent Law Firm LLC, P.O. BOX 4850, FRISCO, CO, 80443-4850, US

(21) Appl. No.: 11/627,380

(22) Filed: Jan. 26, 2007

(51) Int. Cl. A61B 17/062

(52) U.S. Cl. 81/487

(57) ABSTRACT

The invention comprises the body (1), handle (2), moving needle fixing jaw (3), magnet (4), runner (5), lock mechanism (6), lock mechanism spring (7), trigger (8), trigger lock edge (9), the arm transmitting the trigger motion (10), needle fixing control wire (11), needle fixing control wire pin (12), needle fixing control wire pin window (13), trigger spring (14), trigger window (15), runner control head (16), runner control wire pin window (17), runner control wire (18), runner control wire pin (19), runner rotating arm (20) and runner rotating point (21).

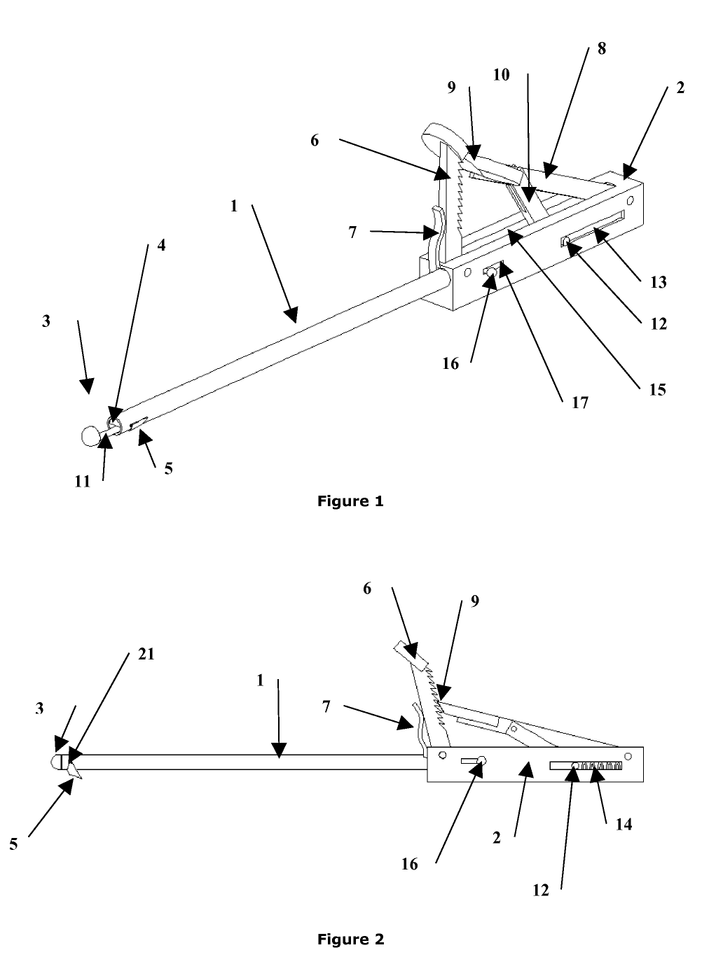

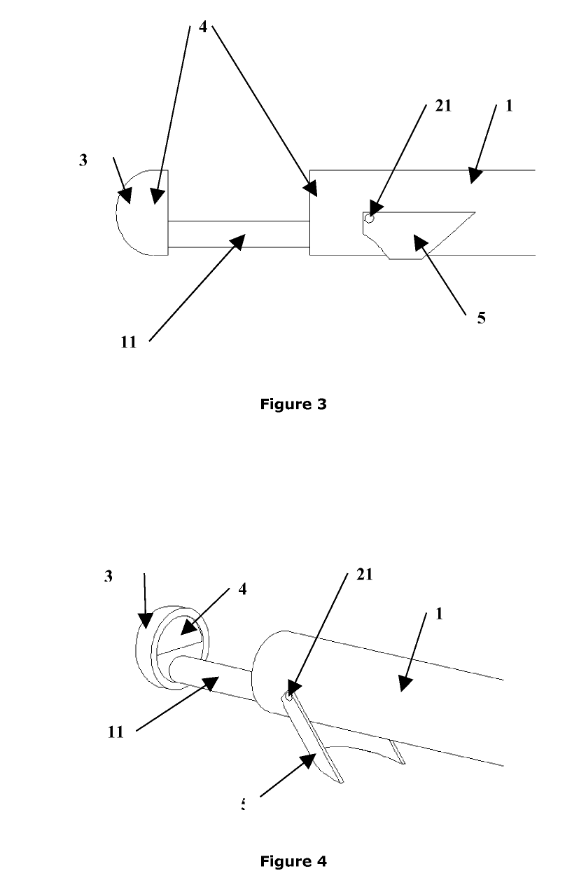

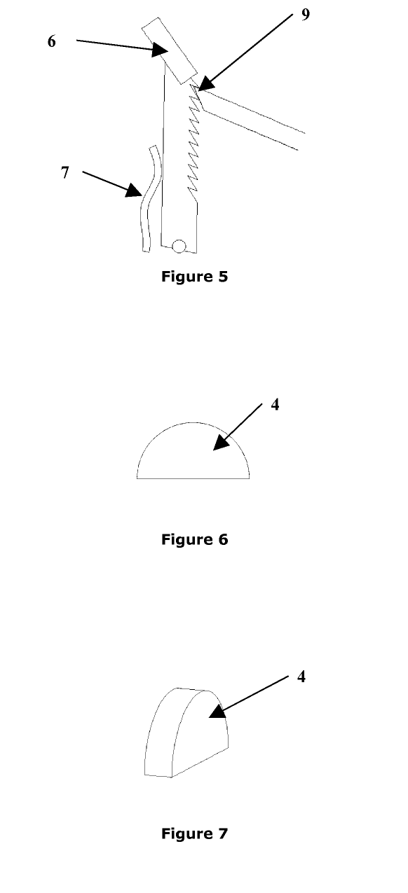

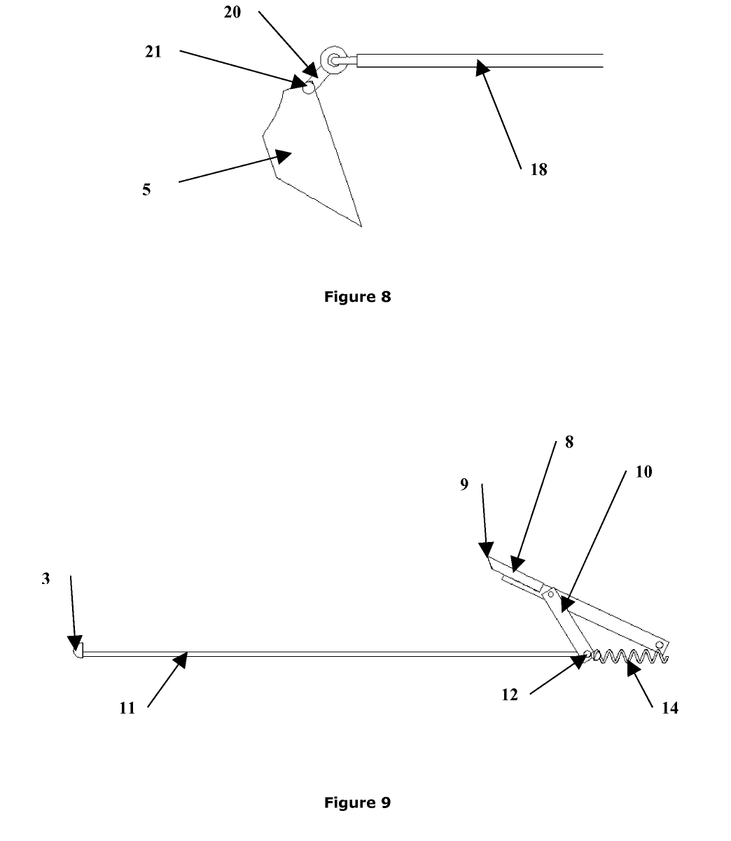

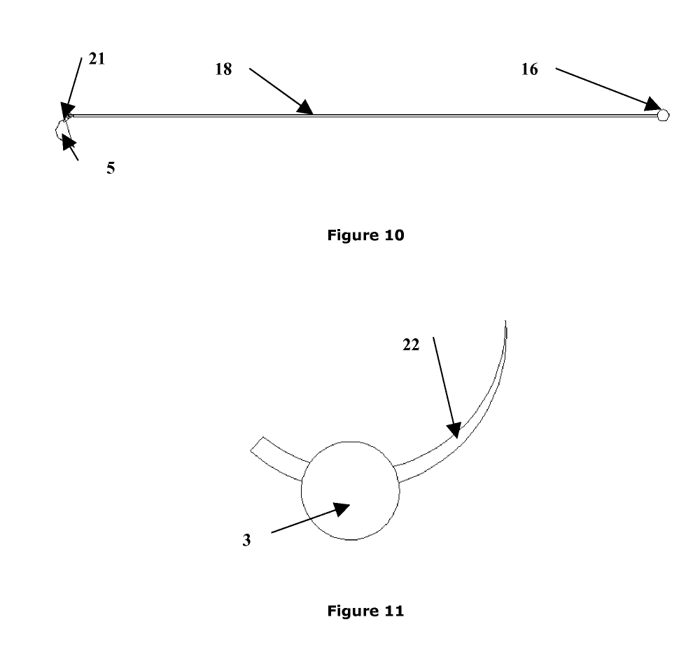

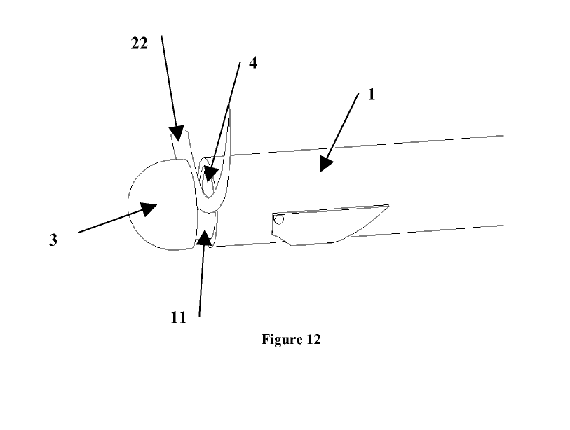

### TECHNICAL FIELD OF THE INVENTION AND THE BACKGROUND ART

The invention relates to suture procedure in laparoscopic surgery. In the current state of the art, this procedure is carried out using the traditional instruments with two jaws.

### TECHNICAL PROBLEMS THE INVENTION AIMS TO SOLVE

Currently, single-camera imaging systems are used in laparoscopic surgery, and this leads to difficulties with the needle holders currently available, during the steps of the suture procedure like capturing the needle and bringing it to suitable position, and to organ injuries due to the opening and closing actions in order to capture the needle with the needle holder jaws. With the invention, the magnet at the end helps the needle to be easily drawn towards between the jaws even from a certain distance and once the needle is located between the jaws, it can be easily given the suture position. With the invention, it has also become easier to grab the yarn. Owing to the invention, it has also been made easier for the two instruments to operate in connection with each other.

### DESCRIPTION OF THE DRAWINGS

1) Perspective view

2) Side view, with runner open and trigger pressed

3) Detail of needle capturing end, side view

4) End part, oblique rear view, runner open

5) Lock mechanism, side view

6) Magnet, front view

7) Magnet, oblique view

8) End detail of runner mechanism (body removed)

9) Needle fixing mechanism (handle, body and runner mechanism removed)

10) Runner mechanism (handle, body and needle fixing mechanism removed)

11) End detail, needle captured, front view

12) End detail, needle captured, slightly oblique view

### PART NUMBERS

1. Body
2. Handle
3. Moving needle fixing jaw
4. Magnet
5. Runner
6. Lock mechanism
7. Lock mechanism spring
8. Trigger
9. Trigger lock edge
10. The arm transmitting the trigger motion
11. Needle fixing control wire
12. Needle fixing control wire pin
13. Needle fixing control wire pin window
14. Trigger spring
15. Trigger window
16. Runner control head
17. Runner control wire pin window
18. Runner control wire
19. Runner control wire pin
20. Runner rotating arm
21. Runner rotating point
22. Needle

### DESCRIPTION OF THE INVENTION

The invention comprises the body (1), handle (2), moving needle fixing jaw (3), magnet (4), runner (5), lock mechanism (6), lock mechanism spring (7), trigger (8), trigger lock edge (9), the arm transmitting the trigger motion (10), needle fixing control wire (11), needle fixing control wire pin (12), needle fixing control wire pin window (13), trigger spring (14), trigger window (15), runner control head (16), runner control wire pin window (17), runner control wire (18), runner control wire pin (19), runner rotating arm (20) and runner rotating point (21).

The pipe body (1) with the diameter of 5 mm. is mounted on one end of the handle (2), which is easy to control by hand. Present at the other end of the body are the moving needle fixing jaw (3) and the magnets (4) placed both inside this jaw and inside the end of the body on this side. At the lower part of the end of the body which contains the magnet, there is located the runner (5) which may be opened when needed and helps positioning on another instrument.

The lock mechanism (6) in the handle is continuously pushed by the lock mechanism spring (7) towards the trigger (8). The trigger lock edge (9), which contacts the lock mechanism at the farthest end, is triangular in shape and is fully compatible with the notches on the lock mechanism. In this way, by the continuous pushing of the lock mechanism spring, the trigger lock edge fits in the notch currently suitable and its backward motion is prevented, in other words, it becomes locked. In order to permit backward motion, that is, to release the trigger, the lock mechanism is pushed in a direction opposite of the trigger. The trigger released from the notch goes up.

The pressing motion of the trigger is transmitted by the arm transmitting the trigger motion (10) to the needle fixing control wire (11). There is located the needle fixing control wire pin (12) at the rearmost end of this wire. This pin moves forwards and backwards inside the needle fixing control wire pin window (13) on the handle, and transforms the downwards pressing action of the trigger into forward-backward motion. As this pin is continuously being pushed by the trigger spring (14) towards the end, this action provides the upwards motion to the trigger via the arm transmitting the trigger motion (10). Said upwards-downwards motions of the trigger take place inside the trigger window (15) located on the upper part of the handle. Thus, the motions of the trigger are transmitted by the needle fixing control wire to the moving needle fixing jaw which is mounted to this wire. When the trigger is pressed, the wire and the moving jaw connected to it are pulled towards the end of the body. Meanwhile, the object between the moving jaw and front end of the body becomes fixed.

The forwards-backwards motions of the runner control head (16) inside the runner control wire pin window (17) are transmitted by the runner control wire pin (19), which is connected both with said head and the runner control wire (18), to the runner control wire. Said wire provides forwards-backwards motion of the runner rotating arm (20) over the runner rotating point (21).

### APPLICATION METHOD OF THE INVENTION

The handle (1) is taken inside the palm. At the initial position, the trigger (8) is open and located above by the backwards pushing of the trigger spring (14). The trigger lock edge (9) is inside the uppermost notch of the lock mechanism (6). In this condition, the needle fixing jaw (3) is away from the body (1) with a distance sufficient to accommodate an object. The runner (5) is closed, as the runner control head (16) is at the foremost position. The instrument is inserted in this way within the 5 mm pipe into the abdomen of the patient, which is inflated by gas, in order to perform suture in endoscopic operation. It is brought close to the needle (22), which has been previously introduced into patient's abdomen through another hole. The magnets (4) at the end attract the needle towards the exact position where it should fit. Then the trigger is pressed. The fixing jaw moving towards the body is both secured and remains stationary at this point, by the trigger lock mechanism fitted in one of the jaw openings. After the suture is performed, the lock mechanism is pushed away from the trigger in order to release the needle. The trigger released from the notch goes up by the pushing action of its spring and releases the needle for the other suture. In case there is another instrument within the operating area, the runner is opened by pulling backwards its head in order to aim at the location where the suture is to be performed, the space below the runner is placed on said other instrument and the instrument is slipped towards the location of suture.

1. The needle holder with magnet characterized in that it comprises the body (1), handle (2), moving needle fixing jaw (3), magnet (4), runner (5), lock mechanism (6), lock mechanism spring (7), trigger (8), trigger lock edge (9), the arm transmitting the trigger motion (10), needle fixing control wire (11), needle fixing control wire pin (12), needle fixing control wire pin window (13), trigger spring (14), trigger window (15), runner control head (16), runner control wire pin window (17), runner control wire (18), runner control wire pin (19), runner rotating arm (20) and runner rotating point (21).

2. The needle holder with magnet according to claim 1 characterized in that the needle fixing jaw (3) and the corresponding end of the body (1) have a round shape and that they contain magnet (4).

3. The needle holder with magnet according to claim 1 characterized in that it has the locking mechanism comprising the lock mechanism (6), lock spring (7) and trigger lock edge (9).

4. The needle holder with magnet according to claim 1 characterized in that it comprises a handle (2) which enables holding in an ergonomic manner.

5. The needle holder with magnet according to claim 1 characterized in that it comprises the moving jaw (3) with such a structure to strongly secure the needle so that it will not rotate in any direction when the same is fixed, and the corresponding body (1) having the same diameter, located opposite said jaw.

6. The needle holder with magnet according to claim 1 characterized in that it comprises the grooved runner (5) which will enable another instrument to be located thereon to be conveyed to the location where the suture is to be performed and that said runner is capable of being opened and closed by means of a head (16) on the handle.
